(19) United States
(12) Patent Application Publication
(10) Pub. No.: US 2009/0228047 A1
(43) Pub. Date: Sep. 10, 2009

(54) Device for reducing a fracture, in particular a distal radius fracture

(76) Inventors: Guillaume Derouet, St. Herblain (FR); Gregoire Larche, Cholet (FR)

Correspondence Address: MCHALE & SLAVIN, P.A., 2855 PGA BLVD, PALM BEACH GARDENS, FL, 33410, US

(21) Appl. No.: 12/352,351

(22) Filed: Jan. 12, 2009

(30) Foreign Application Priority Data
Mar. 7, 2008 (FR) 08/01266

(51) Int. Cl. A61B 17/70; A61B 17/58

(52) U.S. Cl. 606/286; 606/280; 606/301; 606/96; 606/104

(57) ABSTRACT

The device according to the invention, for reducing a fracture, in particular a distal radius fracture, comprises:

an osteosynthesis plate with an elongated body part prolongated by a single-piece head part, said head part including orifices adapted for “poly-axial” assembly of fastening screws,

a guiding block type device fitted with through-orifices adapted for acting as a guide to said fastening screws,

means for drilling orifices with a set of suitable drill bits,

a first drilling guide whereof the threaded insert adapted for being screwed in one of the nuts of the plate head, the latter being deprived of the guiding block, to enable the realisation of an drill-hole in the bone by a “poly-axial” type technique, and

a second drilling guide adapted for co-operating with one of the orifices of said guiding block positioned sur said plate head so as to enable the realisation of a drill-hole in the bone by a “mono-axial” type technique.

The present invention relates a device, or more accurately a set of devices (or kit) offered to surgeons for reducing a bone fracture, in particular a distal radius fracture.

There are numerous systems such as plate(s) associated with a set of fastening screws, offered to surgeons for reducing a bone fracture.

According to the type of fracture at hand, to provide optimal reduction, it may be interesting to tilt the axis of one or of each fastening screw, appropriately, relative to the perpendicular of the supporting plate.

To do so, and for simple fractures, current or relatively current, certain plates comprise “mono-axial” type fastening screws, with a preset orientation. For easy placement of these screws, it is known to use a device called “gun-block” or “drill guiding block”, added removably on the supporting plate, fitted with a plurality of through-orifices, intended each to come in regard of one of the orifices of the supporting plate and whereof the axis is tilted correctly in relation to the tilting requested for the corresponding screw.

This guiding block hence enables a predetermined configuration for the associated fastening screws; it is used, in combination with a so called “drilling gun” or “drilling guide”, for acting as a guide for drilling in the material of the screw implanting drill-holes (surgical motor and drill bit), and for acting as a guide to the screws properly speaking when being placed.

For complex fractures, the surgeon may use plates fitted with so-called “poly-axial” fastening screws, i.e. whereof the implantation angle into the bone can be adjusted along an admissible predetermined tilting range.

According to the case at hand, the surgeon may then select the tilting of the fastening screws which it judges as optimal.

These screws are placed after realisation of a drill-hole into the bone, by means of the drilling device, associated with a guiding gun correctly oriented in advance according to the fastening direction selected.

Moreover, in all cases, regardless whether it is a “mono-axial” or “poly-axial” technique type osteosynthesis implant, it is interesting to lock the fastening screw, once fully inserted into the bone, for optimising the mechanical stability of the osteosynthesis assembly thus obtained and limiting the disassembly risks thereof (migration of the screws . . . ) during the bone consolidation phase, after placing the bone under load.

Thus, in practice, according to the fracture that he wishes to reduce, the surgeon makes a choice between the mono-axial or poly-axial type systems available to him, said choice being made generally during the surgical operation, after reduction of the fracture.

The techniques using the “mono-axial” type systems with guiding block are less costly and quicker to be implemented, however they do not offer surgeons nearly any latitude for implanting the screws.

On the other hand, the “poly-axial” type systems provide the surgeon with a choice for implanting the screws, but they are more costly and require longer implementation time.

The purpose of the present invention is to optimise the surgeon's intervention, in particular for a distal radius fracture, while offering him a set of devices which he may use regardless of the type of fracture at hand, and this, to suit each fastening screw, according to a poly-axial or mono-axial type technique.

According to the invention, the corresponding device comprises:
an osteosynthesis supporting plate including a bottom face and a top face, said bottom face intended for being located against the receiving bone material; this plate comprises an elongated body part, prolongated by a single-piece head part, wherein said body part comprises a plurality of through-orifices and said head part comprises a plurality of through-orifices, which through-orifices of said head part enable to accommodate poly-axial type screws, i.e. capable of being implanted along an admissible predetermined tilting range, said plate still comprising, at the linking zone between its body part and its head part, at least one complementary threaded orifice,
a set of screws for fastening into the bone, intended for being inserted into said through-orifices of the plate body, so-called “body screws”, for attaching said plate body at the surface of the bone, which body screws comprise a head prolongated by a body fitted with a thread for fastening in the bone,
a set of screws for fastening into the bone, intended for being inserted into said through-orifices of the plate head, so-called “head screws”, for attaching said plate head at the surface of the bone,
a guiding block type device intended for being positioned on the top face of said plate head, to enable set tilting positioning of said head screws, which guiding block comprises a plurality of through-orifices, identical in number to those of said plate head, each intended for matching one of said orifices of said plate head, which through-orifices are adapted for acting as a guide for said head screws, and which guiding block still comprises at least one through-orifice intended for being positioned in the extension of said complementary orifice of the plate head, for its removable fixture thereon by means of a suitable fastening screw,
means for drilling orifices, of surgical motor type associated with a set of suitable drill bits,
a first drilling guide, adapted for co-operating with the orifices of said plate head, the latter being deprived of said guiding block, to enable drilling in the bone a drill-hole for receiving said head screws, and
a second drilling guide, adapted for co-operating with the orifices of said guiding block positioned on said plate head, to enable drilling in the bone a drill-hole for receiving one of said head screws.

Preferably the device according to the invention comprises an osteosynthesis plate whereof the through-orifices of the head part are fitted with means provided for locking the head screws on said plate, upon completion of the screwing operation into the receiving bone material.

According to an advantageous embodiment, each of the through-orifices of the head part of the osteosynthesis plate comprises a housing provided for accommodating and holding a nut, wherein each of said nuts is blocked into rotation in its housing relative to said supporting plate and each of said nuts comprises a spherical contact surface capable of co-operating with a complementary spherical contact surface provided in its reception housing, to confer said nut a degree of freedom in said housing, along an admissible predetermined tilting range, to enable tilting of its axis relative to the axis of said reception housing, said nuts hence being of “poly-axial” type. Moreover, the head screws then comprise a head prolongated by a body fitted—with a thread for fastening in the bone, and—with a thread intended for co-operating with the thread of the related plate nut.

Within the framework of this advantageous embodiment:
the first drilling guide is fitted with a threaded insert adapted for being screwed in one of the nuts of the plate head, the latter being deprived of said guiding block, and
said second drilling guide is adapted for co-operating with one of the orifices of said guiding block, abutting against one of said nuts of said plate head.

Preferably still, the guiding block is fitted with a single through-orifice for the fastening thereof on the osteosynthesis plate and it also comprises, on its bottom face intended for contacting the plate head, at least one toe or one recess intended for co-operating with one complementary toe or recess to match the top face of said plate head, to ensure correct centering of said guiding block on said plate head.

According still to another particularity, the device offered to the surgeon comprises a set of positioning spindles or pegs; moreover, the guiding block and the osteosynthesis plate (at the head part thereof, comprise matching orifices for letting through these positioning pegs.

The second drilling guide (used in combination with the guiding block) comprises advantageously a graduation system intended for co-operating with a mark provided on the related drill bit, so as to determine the drilling depth performed, and thus to determine the suitable screw length to be used.

On the other hand, the device available to the surgeon comprises further advantageously a gauge capable of determining the length of the through drill-hole provided through the drilled bone, which gauge comprises a hollow cylindrical body whereof the insert is adapted for resting on the osteosynthesis plate, optionally through one of the orifices of the guiding block, associated with a ram whereof one of the ends is fitted with a graduated system and whereof the other end is fitted with a positioning hook.

The invention will be illustrated further, without being limited thereto, by the following description of a particular operating technique, using the set of devices according to the invention, especially in relation with the appended drawings wherein:

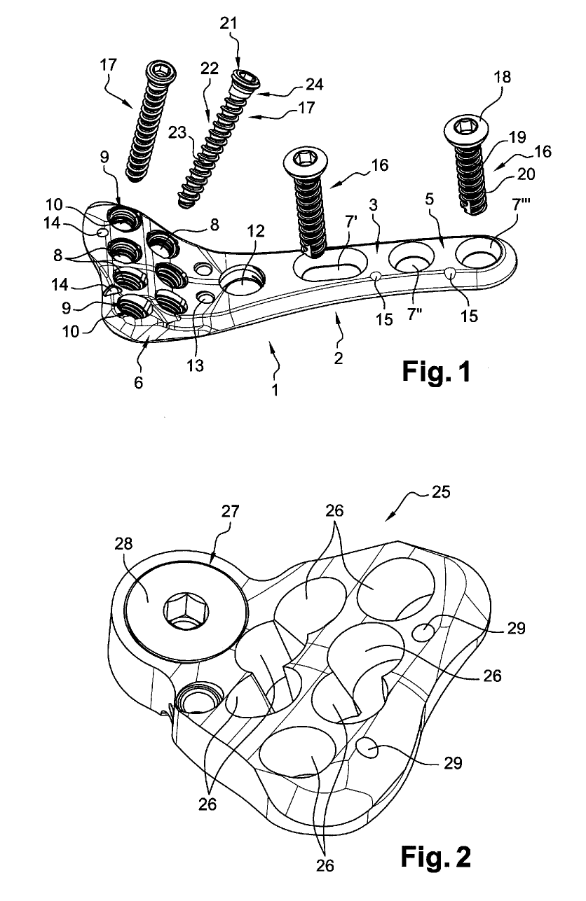

FIG. 1 is a perspective view of the osteosynthesis plate part of the device offered;

FIG. 2 is a perspective view of the guiding block part of the device offered;

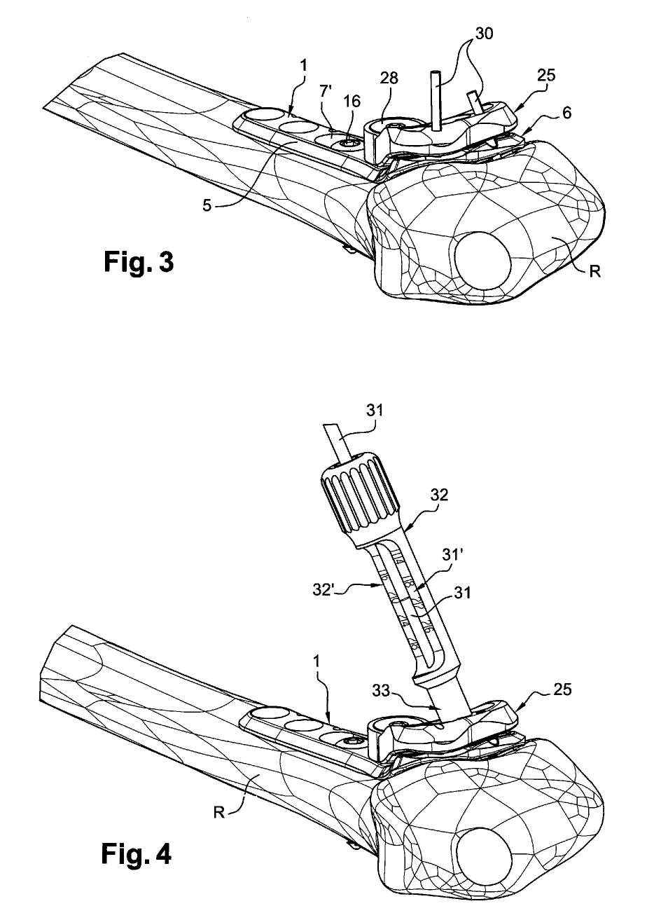

FIG. 3 the plate/guiding block assembly during correct positioning on the end of a radius, in combination with positioning pegs;

FIG. 4 illustrates the drilling of a drill-hole in the end of the radius, for positioning a head screw according to a mono-axial technique using the guiding block and a suitable drilling guide;

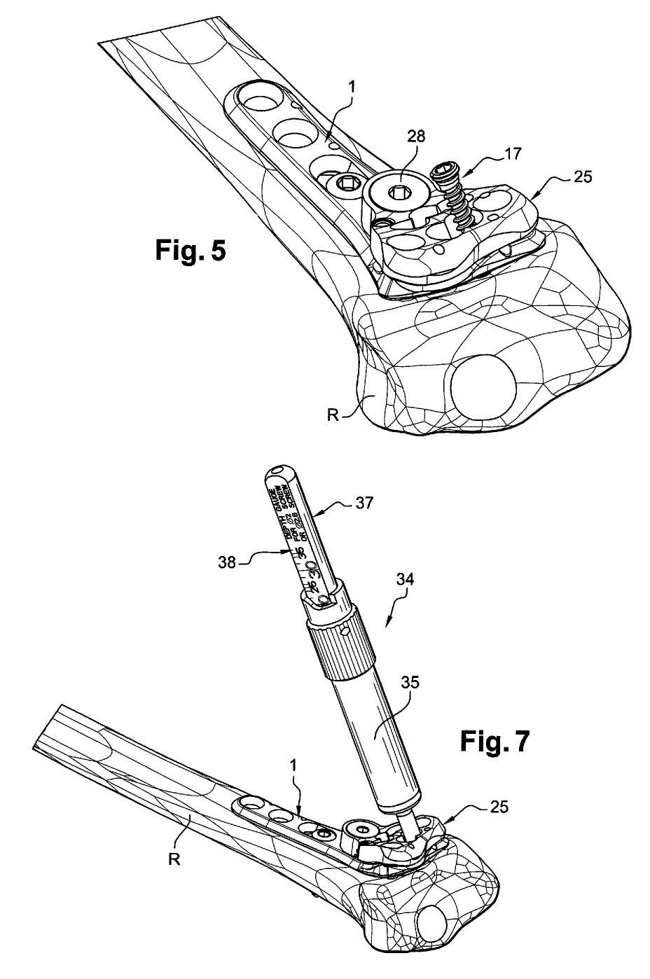

FIG. 5 shows the placement of a head screw through the guiding block, after drilling the drill-hole illustrated on FIG. 4;

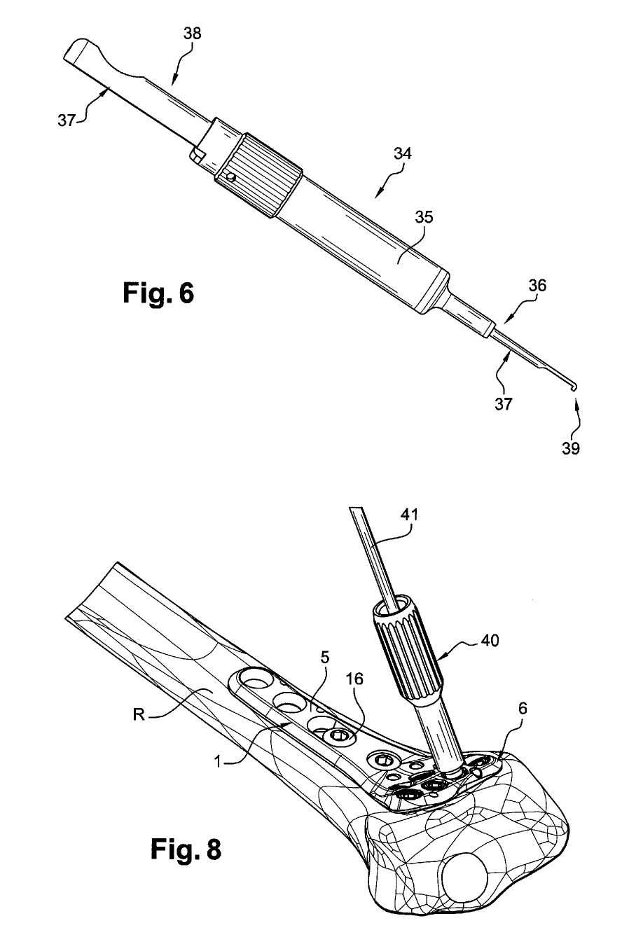

FIG. 6 shows the gauge enabling to determine the length of a through drill-hole provided in the bone;

FIG. 7 shows the use of the gauge illustrated on FIG. 6, on the plate/guiding block assembly;

FIG. 8 shows the use of the other drilling guide for the implementation of the poly-axial fastening technique;

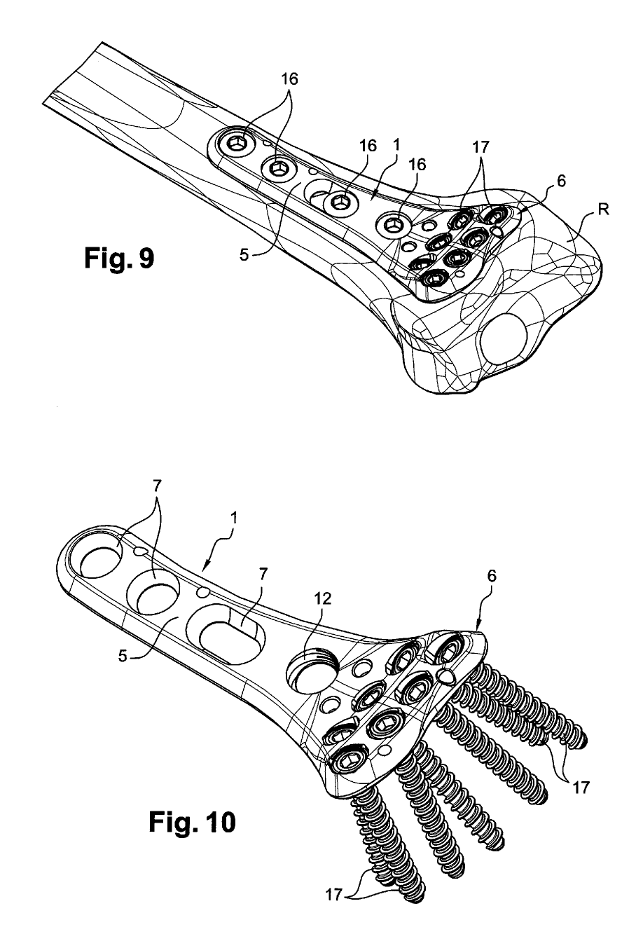

FIG. 9 is a view of the osteosynthesis plate mounted correctly on the radius with the set of its fastening screws;

FIG. 10 is an individual view of the plate, showing the implantation of the different head screws.

As illustrated on the Figures, the device according to the invention offered to the surgeon, for the reduction of a distal radius fracture, comprises in particular an osteosynthesis plate, a set of diaphysial screws, a set of epiphysial screws, a guiding block, two drilling guides, a set of positioning pegs and a drilling device (surgical motor and a set of suitable drill bits).

The osteosynthesis plate 1 illustrated individually on FIG. 1, is adapted for the reduction of a distal radius fracture; it comprises a bottom face 2 intended for contacting the bone material and a top face 3. This plate 1 is formed of an elongated body part 5 suitable for being placed on the diaphysis of the bone, and of a one-piece head part 6, suitable for being placed on the bone epiphysis.

The body part 5 comprises a plurality of aligned through-orifices 7′, 7″, 7′″ (three in this case), whereof one 7′ exhibits an oblong elongated shape. The head part 6 comprises for its own part a plurality of through-orifices 8 provided on two lines substantially perpendicular to the axis of the body part 5. The (end) distal line is composed of an alignment of four orifices 8 whereas the proximal line is composed of an alignment of three orifices 8.

Each head orifice 8 comprises a housing 9 for receiving a “poly-axial” nut 10, for instance as described in Documents FR-A-2 832 308 and FR-07 02777.

This nut 10 comprises a threaded central orifice and a spherical contact surface (not visible on FIG. 1) suitable for co-operating with a complementary spherical surface provided in the reception housing 9 (also not visible on FIG. 1) for conferring its poly-axial character thereto (i.e. for conferring a degree of tilting freedom thereto) along an admissible predetermined tilting range. Tenon/mortise type means are also provided to lock the nuts 10 into rotation in their reception housing 9 (these means do not appear on FIG. 1).

At the linking zone between the body part 5 and the head part 6, the plate 1 comprises a complementary orifice 12 fitted with a thread.

Moreover, on the top face of the head part 6, just in front of the complementary orifice 12, the presence of two small blind mortises 13 in the form of cylindrical reservations can be noted.

The distal end of the head part 6 comprises further two small unthreaded through-orifices 14. On one of the sides of the body part 5, the presence of two small unthreaded through-orifices 15 can also be noted, intended for receiving temporarily and if required, pegs which may hold the plate on the diaphysis before the placement of the fastening screw.

The device according to the invention also comprises a set of screws 16 for attaching on the bone the plate body 5 via through-orifices 7 (so-called “body screws”), and a set of screws 17 for attaching on the bone the plate head 6 via through-orifices 8 (so-called “head screws”). The body screws 16 are each formed of a head 18 prolongated by a body 19 fitted with a bone thread 20. The head screws 17 are each formed of a head 21 prolongated by a body 22 fitted with a bone thread 23 and a thread 24 intended for co-operating with the thread of the orifice of one of the nuts 10.

On the other hand, the set of devices according to the invention also comprises a guiding block type device 25, as illustrated on FIG. 2. This guiding block 25 consists of a block of material having for instance a thickness of the order of 4 to 6 mm, whereof the general shape corresponds approximately to that of the head part 6 of the osteosynthesis plate 1, and which is fitted with through-orifices 26. The number of these through-orifices 26 is identical to that of the orifices 8 of the head part 6 of the plate 1 (i.e., seven) and these orifices 26 are provided each for matching one of these head orifices 8, when said guiding block 25 is mounted on said plate 1.

The guiding block 25 comprises further an orifice 27 intended for being positioned opposite the threaded complementary orifice 12 of the plate 1, for the positioning of a screw 28 enabling the removable fixture thereof on said plate 1. The centered correct positioning of the guiding block 25 on the plate head 6 is obtained by means of toes or of cylindrical single-piece protruding studs (not visible on FIG. 2) located on its bottom face and which are intended for penetrating the complementary reservations 13 aforementioned of the plate head 6.

The through-orifices 26 of the guiding block 25 are cylindrical and unthreaded; they are each intended for acting as a guide for accurate orientation positioning of the head screws 17.

The axis of each of the orifices 26 is particular, oriented along a predetermined direction corresponding to the accurate direction optimum for the head screws 17 intended for being located in the matching head orifice 8, notably for current fractures, encountered relatively frequently.

The guiding block 25 comprises further two small through-orifices 29 intended for matching the end orifices 14 of the plate head 6, as will be seen below, adapted for letting through positioning pegs.

For the reduction of a distal radius fracture of average complexity, the surgeon puts the guiding block 25 on the plate head 6 by means of the fastening screw 28; the associated tenon/reservation assemblies aforementioned provide for centering and correct positioning of both elements relative to one another.

Then, he places as well as possible the osteosynthesis plate 1 on the radius R and he attaches the body part 5 on the bone structure by means of a body screw 16 in the oblong orifice 7′ (FIG. 3).

The surgeon then checks the plate for correct location by inserting two positioning pegs 30 in the couples of orifices 14-29 of the guiding block 25 and of the plate head 6, as well as through the bone R, so as to make sure that they do not emerge into the joint of the radius. It is there a safety enabling to prevent the head screws 17 laid at a later stage from emerging into the joint of the radius.

The corresponding control on the positioning pegs 30 is provided by radiology or brightness amplifier.

If required, the positioning of the plate 1 is modified longitudinally by loosening the body screws 16 slightly before proceeding to a new check.

Once the plate 1 considered as located correctly, the body screw 16 is clamped.

The surgeon then has the possibility of placing at least some of the head screws 17 according to a “mono-axial” technique via the guiding block 25.

For each of the corresponding screws 17, as illustrated on FIG. 4, a drill-hole in the bone is drilled by means of a surgical motor (not represented) fitted with a drill bit 31, in combination with a drilling guide 32. The cylindrical end 33 of the drilling guide 32 is guided by one of the orifices 26 of the guiding block 25 and it is arranged to abut against the matching nut 10 housed in the plate head 6. The cylindrical head 33 of the drilling guide 32 has a diameter corresponding, within the clearance, to the diameter of the orifices 26 of the guiding block 25.

The matching drill-hole is provided until the head of the drill bit 31 reaches the opposite cortical of the bone (for correct hooking of the screw 17 with the longest possible grip).

A mark 31′ on the drill bit 31, associated with a graduation 32′ provided on the drilling guide 32 enable to know accurately the length of the head screw 17 which should then be used.

After removing the drill bit 31 and the drilling guide 32, the corresponding screw 17 is placed (FIG. 5) through the related orifice 26 of the guiding block 25. Once fully inserted, this screw 17 is compressed on the plate 1 and locked by the presence of the related nut 10.

The operation is repeated for all the head screws 17 that the surgeon wishes to implant according to this “mono-axial” technique.

This implemented facilitates substantially the surgeon's work and vastly limit the operating time of the osteosynthesis.

If the surgeon runs completely through the radius R when making the drill-hole for the screws, the device available to him comprises a gauge 34, illustrated individually on FIG. 6, suitable for determining the dimension of the screws 17 to be used.

This gauge 34 comprises a body 35 generally cylindrical and hollow in shape, whereof the head 36 is arranged for running through the corresponding orifice 26 of the guiding block 25 and for resting on the top face 3 of the osteosynthesis plate 1. This gauge 34 comprises further a ram 37, housed in the hollow body 35, whereof the upper portion 38 is graduated and whereof the lower end is formed of a rod fitted with an end hook 39. It should be understood that once the gauge body 35 is resting on the plate 1, the ram 37 may be operated so as to hook the opposite cortical of the bone, through the drill-hole provided, this hooking enabling to determine the exact depth of the drill-hole by means of the graduation 38, facing the upper end of the hollow body 35, to define the adequate screw length (i.e. a screw whereof the end will reach the opposite cortical of the bone). FIG. 7 illustrates the use of this gauge 34 on the plate 1 fitted with the guiding block 25.

Once the surgeon has placed all the screws 17 he wished to install in “mono-axial” technique, he removes the guiding block 25 (simply by loosening the screw 28). It may then place the remaining head screws 17 according to a “poly-axial” technique.

He realises to do so drill-holes by using a drilling guide 40 (FIG. 8) associated with a surgical motor (not represented) fitted with an adapted drill bit 41. The head of the drilling guide 40 is fitted with a thread and it is screwed in the nut 10 associated with the corresponding head orifice 8; the surgeon adjusts the angularity of the gun 40 for making correctly the drill-hole along the requested direction; he makes the drill-hole up to the opposite cortical of the bone and he uses the gauge 34 aforementioned, illustrated on FIG. 6, to determine the suitable screw length to be used.

The surgeon may complete the placement of the head screws 17 while repeating this succession of operations.

The attachment of the osteosynthesis plate 1 is completed by placing the remaining body screws 16. It should be noted here that the complementary orifice 12 of the osteosynthesis plate 1 is used advantageously for fixing a body screw 16 identical to the others.

An osteosynthesis plate 1 laid and attached on the radius R as illustrated on FIG. 9 is obtained.

FIG. 10 illustrates a possible angular configuration of the different head screws 17.

According to the type of fracture at hand, the set of devices according to the invention may also be used by the surgeon for laying all the head screws 17 in mono-axial technique, by using solely the guiding block 25 (in particular for simple fractures), or for laying the set of head screws 17 in poly-axial technique, by using solely the drilling guide 40 (in particular for very complex fractures).

When he wishes to use both techniques, the surgeon starts preferably with the mono-axial technique so as to use a guiding block 25 pre-positioned on the osteosynthesis plate 1 and so as to use the poly-axial technique on an osteosynthesis plate 1 already secured in position on the bone.

1. A device for reducing a fracture, in particular a distal radius fracture, characterised in that it comprises, in combination:
an osteosynthesis supporting plate (1) including a bottom face (2) and a top face (3), said bottom face (2) intended for being located against the receiving bone material (R), which plate (1) comprises an elongated body part (5), prolongated by a one-piece head part (6), said body part (5) including a plurality of through-orifices (7) and said head part (6) including a plurality of through-orifices (8), which through-orifices (8) of said head part (6) enabling to accommodate poly-axial type screws, i.e. capable of being implanted along an admissible predetermined tilting range, said plate (1) still comprising, at the linking zone between the body part (5) and the head part (6), at least one complementary threaded orifice (12),
a set of screws (16) for fastening into the bone, intended for being inserted into said through-orifices (7) of the plate body (5), so-called “body screws”, for attaching said plate body (5) to the surface of the bone, which body screws (16) comprise a head (18) prolongated by a body (19) fitted with a thread (20) for fastening into the bone,
a set of screws (17) for fastening into the bone, intended for being inserted into said through-orifices (8) of the plate head (6), so-called “head screws”, for attaching said plate head (6) to the surface of the bone,
a guiding block type device (25) intended for being positioned on the top face (3) of said plate head (6), to enable set tilting positioning of said head screws (17), which guiding block (25) comprises a plurality of through-orifices (26), identical in number to those (8) of said plate head (6), each intended for matching one of said orifices (8), which through-orifices (19) are adapted for acting as a guide for said head screws (17), and which guiding block (25) still comprises at least one through-orifice (27) intended for being positioned in the extension of said complementary orifice (12) of the plate head (6), for its removable fixture thereon by means of an adapted fastening screw (28),
means for drilling orifices, of surgical motor type associated with a set of suitable drill bits (31, 41),
a first drilling guide (40), adapted for co-operating with the orifices (8) of said plate head (6), the latter being deprived of said guiding block (25), to enable drilling into the bone a drill-hole for receiving said head screws (17),
a second drilling guide (32), adapted for co-operating with the orifices (26) of said guiding block (25) positioned on said plate head (6), to enable drilling into the bone a drill-hole for receiving said head screws (17).

2. A device according to claim 1, characterised in that it comprises an osteosynthesis plate (1) whereof the through-orifices (8) of the head part (6) are fitted with means for locking the head screws (17) on said plate (1), upon completion of the screwing operation into the receiving bone material.

3. A device according to claim 2, characterised in that each of the through-orifices (8) of the head part (6) of the osteosynthesis plate (1) comprises a housing (9) provided for accommodating and holding a nut (10), each of said nuts (10) being blocked into rotation in its housing (9) relative to said supporting plate (1) and each of said nuts (10) comprising a spherical contact surface capable of co-operating with a complementary spherical contact surface provided in its reception housing (9), to confer said nut (10) a degree of freedom in said housing (9), along an admissible predetermined tilting range, to enable tilting of its axis relative to the axis of said reception housing (9), said nuts (10) hence being of “poly-axial” type, and in that the head screws (17) comprise a head (21) prolongated by a body (22) fitted—with a thread (23) for fastening into the bone, and with a thread (24) intended for co-operating with the thread of the related plate nut (10).

4. A device according to claim 3, characterised in that:
said first drilling guide (40) is fitted with a threaded insert adapted for being screwed in one of the nuts (10) of the plate head (6), the latter being deprived of said guiding block (25), and
said second drilling guide (32) is adapted for co-operating with one of the orifices (26) of said guiding block (25), abutting against one of said nuts (10) of said plate head (8).

5. A device according to any of the claims 1 to 4, characterised in that it comprises a guiding block (25) fitted with a single through-orifice (27) for the attachment thereof on the osteosynthesis plate (1) and fitted, on its bottom face intended for contacting the plate head (6), with at least one toe or one recess intended for co-operating with one complementary toe or recess (13) provided to match on the top face (3) of said plate head (6).

6. A device according to any of the claims 1 to 5, characterised in that it comprises a set of positioning pegs (30), and in that the guiding block (25) and the plate head (6) comprise matching orifices (14, 29) for letting through said positioning pegs (30).

7. A device according to any of the claims 1 to 6, characterised in that the second drilling guide (32) comprises a graduation system (32′) intended for co-operating with a mark (31′) provided on the related drill bit (31), so as to determine the drilling depth performed, and thus to determine the suitable screw length (17) to be used.

8. A device according to any of the claims 1 to 7, characterised in that it comprises a gauge (34) capable of determining the length of the through drill-hole provided through the drilled bone, which gauge (34) comprises a hollow cylindrical body (35) whereof the insert (36) is adapted for resting on the osteosynthesis plate (1), optionally through one of the orifices (26) of the guiding block (25), associated with a ram (37) whereof one of the ends is fitted with a graduated system (38) and whereof the other end is fitted with a positioning hook (39).
